(19) United States
(12) Patent Application Publication
(10) Pub. No.: US 2025/0312357 A1
(43) Pub. Date: Oct. 9, 2025

(54) PROCESS OF PHYTOCANNABINOIDS ENRICHMENT ITS FORMULATION AND USE IN THE MANAGEMENT OF PAIN

(71) Applicant: COUNCIL OF SCIENTIFIC & INDUSTRIAL RESEARCH, New Delhi (IN)

(72) Inventors: Pankaj Singh CHAM, Jammu (IN); Manmeet SINGH, Jammu (IN); Aman VERMA, Jammu (IN); Radhika ANAND, Jammu (IN); Rohit SINGH, Jammu (IN); Kuhu SHARMA, Jammu (IN); Pankul KOTWAL, Jammu (IN); Abhishek GOUR, Jammu (IN); Pankaj KUMAR, Jammu (IN); Durga Prasad MINDALA, Jammu (IN); Sumit ROY, Jammu (IN); Chandra Pal SINGH, Jammu (IN); Siya Ram MEENA, Jammu (IN); Ajay KUMAR, Jammu (IN); Mahendra Kumar VERMA, Jammu (IN); Vishav Prakash RAHUL, Jammu (IN); Utpal NANDI, Jammu (IN); Sumeet GAIROLA, Jammu (IN); Anil Kumar KATARE, Jammu (IN); Deepika SINGH, Jammu (IN); Pyare Lal SANGWAN, Jammu (IN); Dhiraj VYAS, Jammu (IN); Sanghapal Damodhar SAWANT, Jammu (IN); Gurdarshan SINGH, Jammu (IN); Vishwakarma Ram ASREY, Jammu (IN); Dumbala Srinivasa REDDY, Jammu (IN); Parvinder Pal SINGH, Jammu (IN)

(73) Assignee: COUNCIL OF SCIENTIFIC & INDUSTRIAL RESEARCH, New Delhi (IN)

(21) Appl. No.: 18/866,360

(22) PCT Filed: May 16, 2023
(86) PCT No.: PCT/IN2023/050459

§ 371 Date: Nov. 15, 2024

(30) Foreign Application Priority Data
May 17, 2022 (IN) 202211028450

(51) Int. Cl. A61K 31/00; A61K 47/36; A61P 29/00; C07C 37/72; C07D 311/80

(52) U.S. Cl. CPC A61K 31/658; A61K 47/36; A61P 29/00; C07C 37/72; C07D 311/80

(57) ABSTRACT

The present invention relates to the process of enrichment of Phytocannabinoids namely CBD and Δ⁹-THC from the dried aerial part (leaves and inflorescence) of Cannabis sativa. The present invention also relates to the preparation of formulation having enriched CBD and THC by combining with appropriate acceptable excipients such as nutriose, dextrin and maltodextrin. The present invention also discloses the use of the phytocannabanoids enriched blend for the management of pain including cancer pain & general pain.

### FIELD OF THE INVENTION

The present invention relates to the process of enrichment of Phytocannabinoids namely CBD and THC from the dried aerial part (leaves and inflorescence) of Cannabis plant. The present invention also relates to a composition of formulation comprising of enriched fractions of CBD and THC and its use for the effective management of pain.

### BACKGROUND OF THE INVENTION

Cannabis has been associated with Indian culture and medicine since centuries; however, due to its misuse as psycho-active substance it was banned worldwide 1980s onwards and put under narcotic list but slow research still continued by several research groups.

Till date, four drugs namely Sativex (nabiximols), Marinol (Dronabinol), Nabilone (Cesamet) and Epidiolex (cannabidiol) has been approved by regulatory bodies and other in advanced clinical trials namely Ajulemic acid (Resunab, Phase-II), Dexanabinol (HU-211 or ETS2101, Phase-I)

Molecules, 2020, 25, 1567, discloses phytocannabinoids as a unique class of compounds majorly occur in cannabis and responsible for most of its medicinal properties and there are >100 such compounds are reported in cannabis. Among the phytocannabinoids, Δ⁹-Tetrahydrocannabinol (Δ⁹-THC) and cannabidiol (CBD) are two majorly studied chemical constituents and studied in depth and also point of focus all around the world. Δ⁹-THC possesses diverse medicinal properties including the suppression of psycho-activity.

Cannabis Cannabinoid Res., 2016, 1.1, 102 & J. Pharmacol., 2007, 152, 984, discloses that Δ⁹-THC is psychoactive & also possessed beneficial effects against number of ailments, and CBD on the other hand is non-psychoactive.

Considering its medicinal potential, cannabis is highly exploited under both herbal drug (also known as botanical, phytopharmaceutical) and modern medicine (New Chemical Entities) categories and number of preparation and products are available in the market (some of those mentioned above also) throughout the globe for curing and management of number of ailments.

Drug development belongs to the botanical and/or phytopharmaceutical drug pathway requires fractions enriched with active constituents, which in cannabis plant are Δ⁹-THC and CBD. Cannabinoids, 1970, 33, 453, disclosed a method for the preparation of cannabis concentrates as well as enriched and pure cannabinoids, which involved repetitive extraction with ethanol followed by repetitive partition with hexane: water system followed by with 50% aqueous alcohol. The organic phase was pooled up and dried and the obtained extract was either taken up for molecular distillation or chromatography using florisil to get the fractions with different strength of phytocannabinoids.

U.S. Pat. No. 6,403,126 describes method of extracting cannabinoids, cannflavins, and/or essential oils from hemp and/or of producing a whole hemp extract lacking Δ⁹-THC. U.S. Pat. No. 6,403,126 used hemp variety which lacks Δ⁹-THC. The steps involes towards the isolation are extraction followed by column chromatography. This application involves the isolation of cannabidiol (CBD) and related compounds but not Δ⁹-THC because the plant variety used is devoid of Δ⁹-THC.

WO2005/061480A1 describes a preparative separation process wherein (-)-Δ⁹-trans-tetrahydrocannabinol is separated from a mixture of cannabinoids in which at least one chromatographic step wherein a mobile phase passes through a stationary phase. The stationary phase comprises a derivatised polysaccharide and the mobile phase comprises carbon dioxide.

US2008/0103193A1, describes another attempt regarding the preparation of enriched fractions involved the Supercritical method by using genetically stable cannabis lines known to produce particular phytocannabinoids. These processes have been successfully demonstrated in large scale by taking these modified cannabis germlines know to either produce Δ⁹-THC or CBD. However, these germlines were grown in special polyhouse/closed conditions and require the control of several parameters of environment and soil, which makes the whole process, cumbersome & somewhat costlier.

J Pharm Belg., 1974, 29, 415 describes the isolation of cannabidiol (I), tetrahydrocannabinol (II) and cannabinol (III) and other related constituent from C. sativa using column chromatography over SiO₂ with 50:50:0.2:0.5 CHCl₃-petroleum ether-DMR-MeOH development solvent.

US2020/108044A1 describes the invention relates to a method for the chromatographic purification of cannabinoid compound using silica particles comprising of amino and/or diol groups and eluting with ethanol and heptanes solvent system.

CA2,549,399 describes the preparative separation process wherein (-)-Δ⁹-trans-tetrahydrocannabinol is separated from a mixture of cannabinoids. The process comprises at least one chromatographic step wherein a mobile phase passes through a stationary phase. The stationary phase comprises a derivatised polysaccharide and the mobile phase comprises carbon dioxide.

US2020/0039908A1 describes the process for the isolation and purification of cannabinoids from Cannabis plant material of different varieties by extracting the plant material using optionally an organic solvent of supercritical fluid with or without modifier.

CA'399 & US'908 describes the separation of cannabinoids using super critical fluid (using CO2), which require specialized apparatus and expertise in handling.

In prior art, there is no large scale commercial feasible method available for the preparation of enriched fractions comprising of either Δ⁹-THC and CBD from the common cannabis to cater the demand of botanical and phytopharmaceutical drug development pathway.

The present invention has developed a feasible process for the enrichment of Δ9-THC and CBD. The optimized process was demonstrated in kilogram scale.

The obtained Δ9-THC and CBD enriched fractions were further mixed in different strength and formulated as well as explored for the medical application in the of management of pain.

### OBJECTIVE OF THE INVENTION

The main objective of the present invention is to provide a process for the preparation of enrich fractions of THC and CBD from the aerial parts of Cannabis Sp.,

Another objective of the invention is to provide a process for the preparation of a blend containing both CBD and THC in the range of 5% to 49.5%

Another objective of the invention is to provide a pharmaceutical formulation comprising a therapeutically effective amount of CBD and THC with different strength ranging from 2.5 to 20% and at least one pharmaceutically acceptable excipient or a vehicle composition of the enrich fractions of Δ⁹-THC and CBD contained formulation.

### SUMMARY OF THE INVENTION

Accordingly, the invention provides a process for the preparation of phytocannabinoids such as (CBD) and (Δ⁹-THC)-enriched fractions from germplasm Cannabis sativa comprising the steps of:
a. repetitive extraction of dried aerial parts of Cannabis sativa by using polar solvent in the ratio of 1:8 (plant material: solvent (w/v));
b. treating or adsorbing the obtained extract from step-a with solid support (silica);
c. extracting the compound(s) adsorbed on silica in step-b with a mixture of polar or/and non-polar solvents;
d. repetitive column chromatography using solid support (silica having different mesh sizes) and eluting the system comprising of mixture of polar & and non-polar solvents.

The present invention provides an improved process for the preparation of pure phytocannabinoids (CBD) and (Δ⁹-THC) from Cannabis sativa comprising the steps of:
a. repetitive extraction of dried aerial parts of Cannabis sativa by using polar solvent in the ratio of 1:8 (plant material: solvent (w/v));
b. treating or adsorbing the obtained extract from step-a with silica gel solid support of 60-120 mesh size;
c. extracting the adsorbed silica of step b with a mixture of polar and non-polar solvents at a proportions of 1:9 (v/v) and collecting the organic layer;
d. column chromatography of the organic layer obtained in step c using silica gel solid support of 100-200 mesh size, eluting with ethyl acetate and hexane at a proportion of 1-5% (v/v) to obtain different fractions;
e. collecting the fraction obtained from step d which contains equal proportion of CBD and Δ⁹-THC content and further enriching it by column chromatography using silica gel solid support of 100-200 mesh size, eluting with ethyl acetate and hexane at a proportion of 1-2% (v/v) of to obtain different fractions;
f. collecting the fractions separately obtained from step d & e and further enriching the CBD enriched fraction and Δ⁹-THC enriched fraction separately with ethyl acetate and hexane solvent system through combi flash chromatography by using normal phase to obtain different fractions and
g. collecting the desired phytocannabinoid enriched fractions separately and further enriching through semi preparative HPLC using reverse phase column (RP-C18) with mixture of acetonitrile and water by isocratic technique to obtain pure CBD and Δ⁹-THC.

In an embodiment of the present invention the polar solvent used in the extraction step is selected from a group consisting of hexane, ethyl acetate, dichloromethane, chloroform, isopropyl alcohol, methanol, ethanol, ethyl acetate/ethanol, ethyl acetate/methanol, chloroform/ethanol and water/ethanol.

In a preferred embodiment the polar solvent is ethanol.

In an embodiment of the present invention the extraction of adsorbed silica with polar and non-polar solvent mixture is repeated for 2 to 4 times.

In an embodiment of the present invention the polar and non-polar solvent mixture used in the extraction of adsorbed silica is selected from group consisting of ethyl acetate: hexane, dichloromethane: hexane and chloroform: hexane.

In an embodiment of the present invention the polar and non-polar solvent mixture is ethyl acetate: hexane.

In an embodiment of the present invention the purity of CBD is 99.0% (w/w) and purity of Δ⁹-THC is purity 98.2% w/w.

The present invention also provides a process for the preparation of a phytocannabinoids enriched blend containing CBD and Δ⁹-THC in the range of 5% to 49.5% comprising the steps of:
a. combining CBD and THC pure fractions as obtained in the above mentioned process;
b. mixing the combined pure fraction of step-a with organic solvent, selected from ethyl acetate, methanol, ethanol and iso-propanol; and
c. drying the pure fraction obtained from step b to obtain the enriched blend.

The present invention also provides a pharmaceutical formulation comprising 2.5 to 20% of enrich blend of CBD and Δ⁹-THC and at least 20 to 30 weight equivalent of one pharmaceutically acceptable excipient or a vehicle for management of pain.

The present invention provides a method of treatment of a condition or disease selected from the group consisting of management of cancer pain and general pain, comprising administering to a patient in need thereof a therapeutically effective amount of enrich fractions of CBD and Δ⁹-THC with different strength ranging from 2.5 to 20%.

### BRIEF DESCRIPTION OF THE DRAWINGS

FIG. 1a: Flow chart of the process of phytocannabinoids enrichment using different solvents.

FIG. 1b: Shows the graphical representation the percentage of phytocannabinoids in extract in different solvents.

FIG. 2a: Flowchart of the process of phytocannabinoids enrichment using different solid support.

FIG. 2b: Shows the graphical representation the percentage of phytocannabinoids in extract using different solid supports.

FIG. 3a: Flowchart of the process of phytocannabinoids enrichment in cannabis extract with mixture of polar and non-polar solvents.

FIG. 3b: Shows the graphical representation the percentage of phytocannabinoids in extract with mixture of polar and non-polar solvents.

FIG. 4a: Flow chart of the process of phytocannabinoids enrichment and isolation from alcoholic extraction.

FIG. 4b: Shows the graphical representation the percentage of phytocannabinoids in extract.

FIG. 5a: Flow chart of the process of phytocannabinoids enrichment and isolation from alcoholic extraction.

FIG. 5b: Shows the graphical representation the percentage of phytocannabinoids in extract.

FIG. 6: Graphical representation the percentage of phytocannabinoids contents after making composition ratio of CBD-5 and THC-5 in extract.
a: % w/w of CBD and THC in CS-016-B1, b: % w/w of CBD and THC in CS-016-B2, c: % w/w of CBD and THC in CS-016-B3, d: % w/w of CBD and THC in CS-017-B1

FIG. 7: In-vivo efficacy study in cancer pain model using the randall selitto test for assessment of mechanical hyperalgesia in male BALB/c mice.

FIG. 8: In-vivo efficacy study in pain model using the tail flick Assay for assessment of thermal nociceptive response in female BALB/c mice.

FIG. 9: In-vivo efficacy for the assessment of pain using acetic acid induced writhing in Female BALB/c Mice.

### Abbreviation

ANOVA=Analysis of Variance
CBN=Cannabinol
CBG=Cannabigerol
CBD-5=Cannabidiol
CVDV=Cannabidivarin
CBDA=Cannabidiolic acid
CBGA=Cannabigerolic acid
C=Chloroform
CPCSEA=Committee for the Purpose of Control and Supervision of Experiments on Animals
CSIR-IIIM=Council of scientific and industrial and research-Indian Institute of Integrative Medicine
CI=Clearance
D=Dichloromethane
Et=Ethanol
Ea=Ethyl acetate
E.V=Extractive value
HPLC=High performance liquid chromatography
H=Hexane
I=Isopropyl alcohol
IAEC=Institutional Animal Ethics Committee
LOD=Limit of Detection
LOQ=limit of Quantification
Me=Methanol
PT=paclitaxel
RP-18=Reverse phase column
SD=Standard deviation
SEM=standard error mean
OSD=Oral solid dosage forms
THC-5=Tetrahydrocannabidiol
THCV=Tetrahydrocannabivarin
THCA=Tetrahydrocannabinolic acid
TLC=Thin layer chromatography
W=Water

### DETAILED DESCRIPTION OF THE INVENTION

While describing specific embodiment of the invention, it is not meant to be restricted to described methods, and experimental conditions, which may vary. The present invention starts with the availability of botanical raw material which may consist of one or more phytocannabinoids.

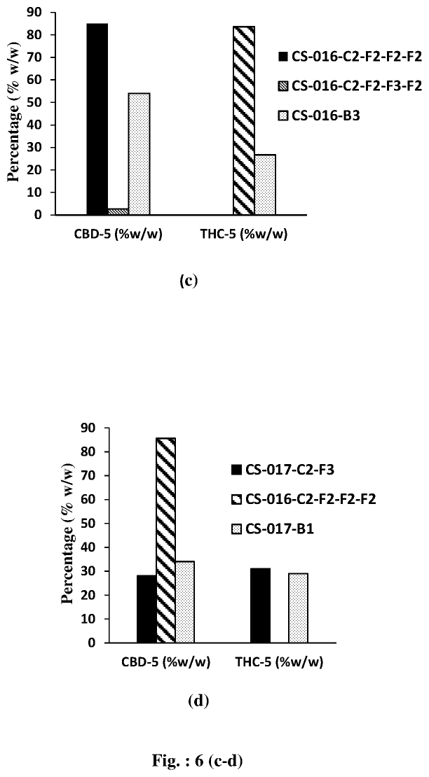

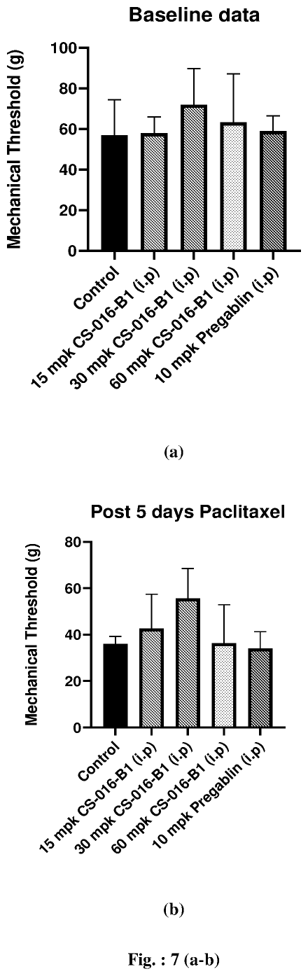

In the present invention the term “Cannabis plant” refers to plants obtained from captive cultivation of Cannabis sativa. The plant material was standardized by the four markers namely CBD, Δ⁹-THC, cannabidiolic acid (CBDA) and Δ⁹-tetrahydrocannabinolic acid (THCA). The results are depicted in FIG. 4b.

In one embodiment, the present invention provides a process for the preparation of phytocannabinoids such as CBD and Δ⁹-THC enriched fractions from germplasm grown in the dedicated and approved area of CSIR-IIIM, Jammu (Chatha Farm, Northern part of India) in normal environmental and soil conditions.

The process of enrichment of phytocannabinoids namely CBD and THC from the dried aerial part (leaves and inflorescence) of Cannabis sativa involves the following steps:
a. repetitive extraction of dried aerial parts of Cannabis sativa by using polar solvent in the ratio of 1:8 (plant material:solvent (w/v));
b. treating or adsorbing the obtained extract from step-a with solid support (silica);
c. extracting the compound(s) adsorbed on silica in step-b with a mixture of polar or/and non-polar solvents and
d. repetitive column chromatography using solid support (silica having different mesh sizes) and eluting the system comprising of mixture of polar & and non-polar solvents.

Steps b and c remove the polar compounds and enriched the required phytocannabinoids.

In an embodiment, the present invention provides an improved process for the preparation of phytocannabinoids such as (CBD-5) and (Δ⁹-THC)-enriched fractions from germplasm (Cannabis sativa) of Indian origin comprising the steps of:
a. repetitive extraction of dried aerial parts of Cannabis sativa by using polar solvent in the ratio of 1:8 (plant material: solvent (w/v));
b. treating or adsorbing the obtained extract from step-a with solid support (silica);
c. extracting the compound(s) adsorbed on silica in step-b with a mixture of polar or/and non-polar solvents;
d. repetitive column chromatography using solid support (silica having different mesh sizes) and eluting the system comprising of mixture of polar & and non-polar solvents.

The solvent is chosen from the group of polar or non-polar organic solvents either alone or in combination thereof. Polar organic solvent is chosen from the group of methanol, ethanol, isopropanol, ethyl acetate and the like; non-polar organic solvent is selected from the group of hexane, heptane, toluene, chloroform, dichloromethane, toluene and the like. In the first step, different solvents were used to extract the phytocannabinoids and the selection of the same for the next step is the decided by the number of criteria such as extractive value, phytocannabinoids percentage and most acceptable solvent. Among all the solvent, ethanol is fulfilling the maximum criteria and therefore, the ethanol based extract has been selected for further step. The use of ethanol is only representative. Extract with other solvents could be used and the similar cascade could be followed for the further enrichment and isolation. Showing ethanol as an example does not restricted the present invention with ethanol, and other can be exploited by the similar way.

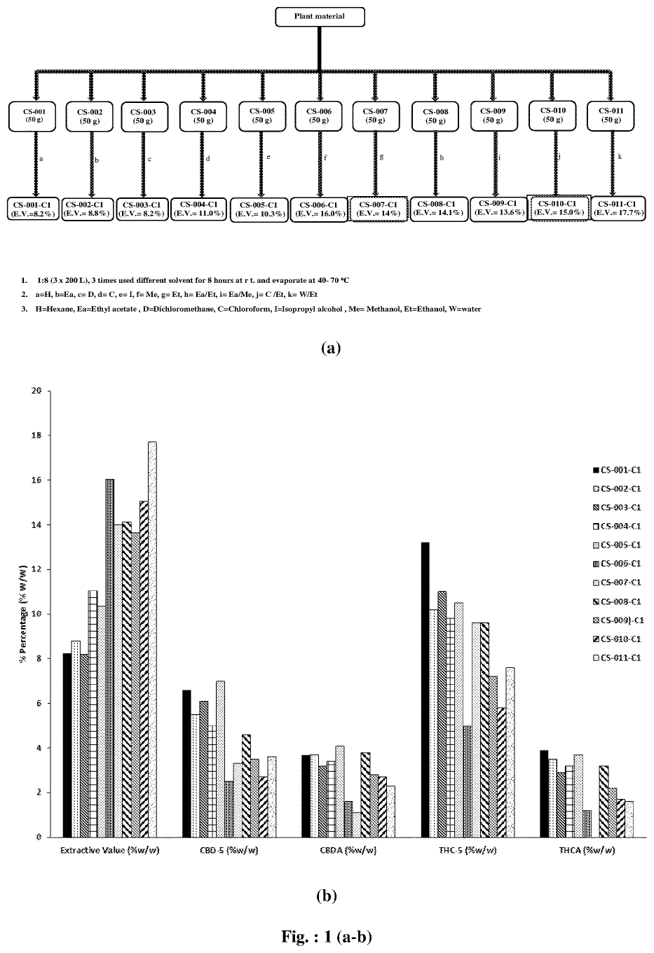

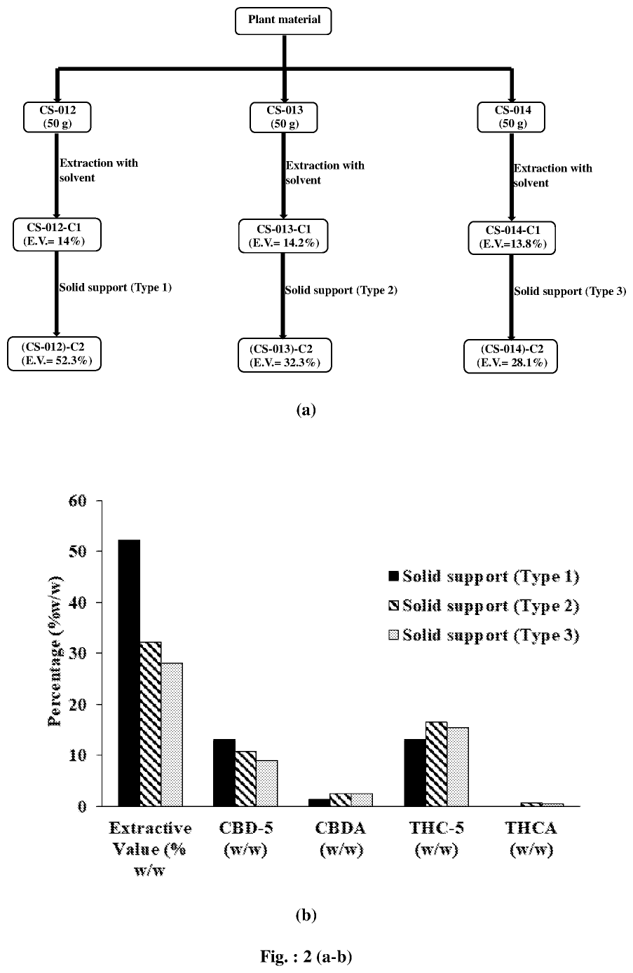

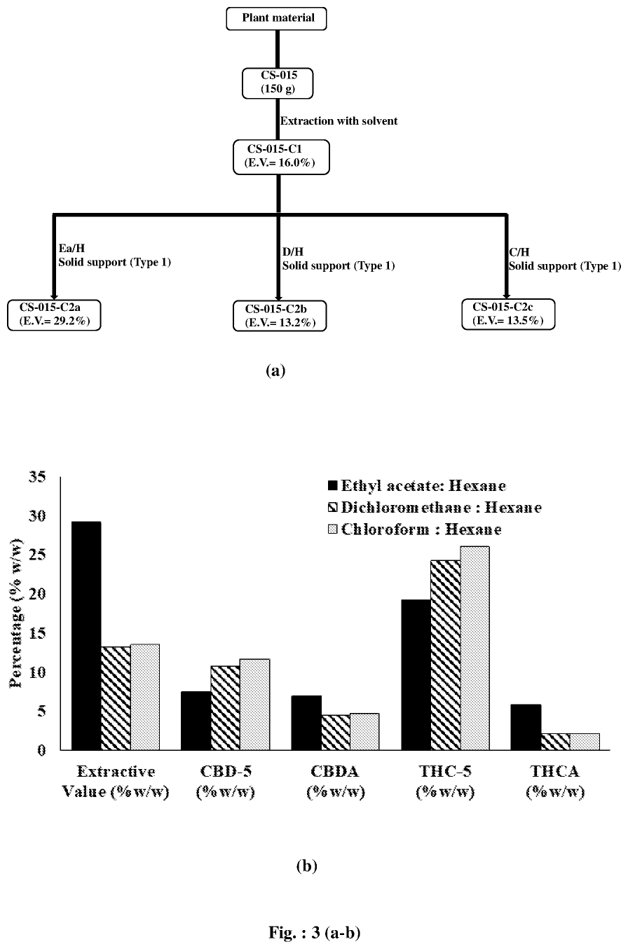

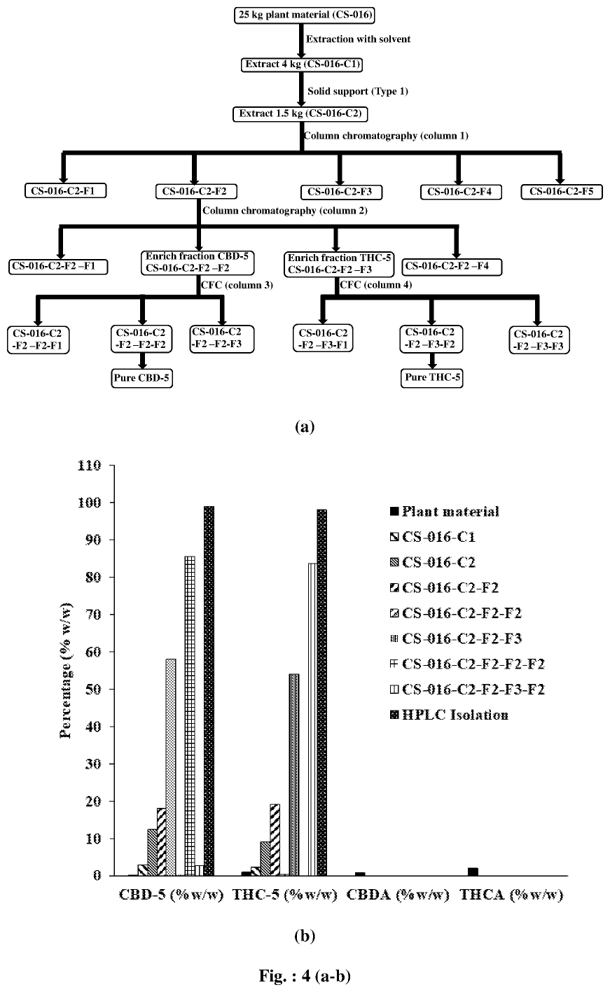

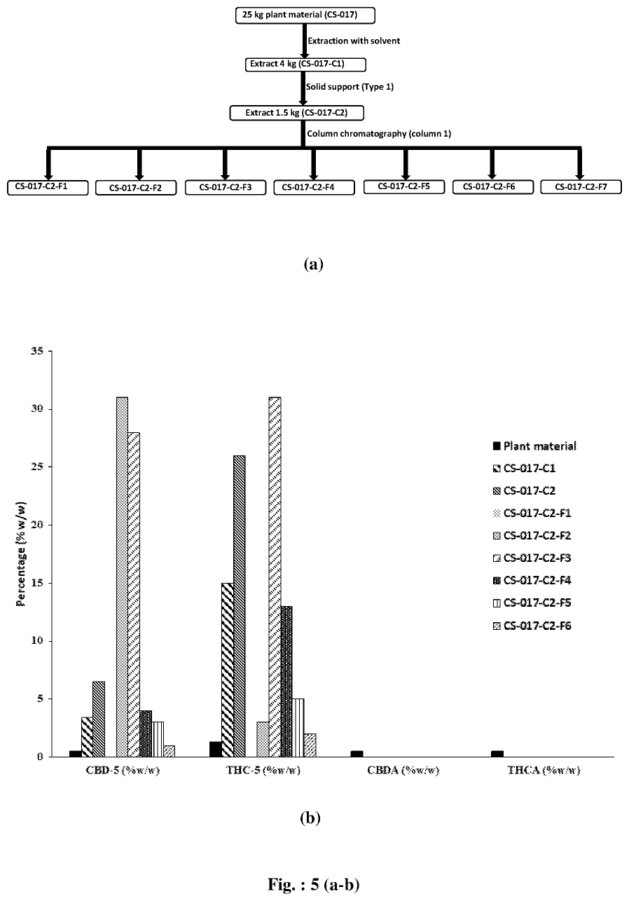

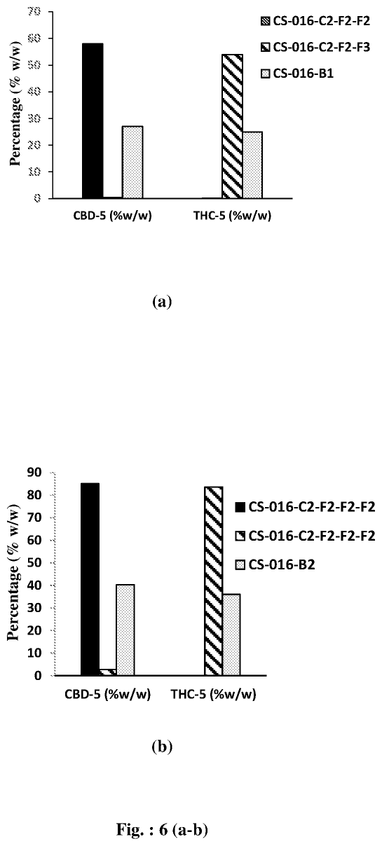

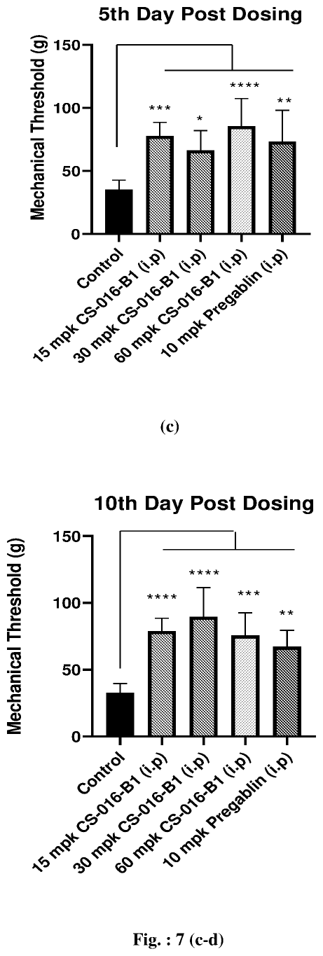

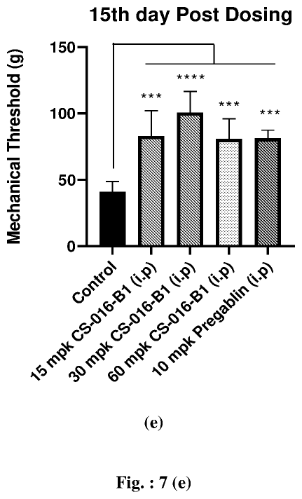

The obtained extract in step-a is labelled as (CS-001-C1 to CS-017-C1), extractive value is in the range of 8% to 17%. The results are shown in FIG. 1b, FIG. 2b, FIG. 3b, FIG. 4b and FIG. 5b. In order to remove the pigments and polar constituents, the obtained extract (CS-001-C1 to CS-017-C1) from step-a was treated with solid supports as depicted in FIG. 2a and FIG. 3a. The extracts obtained in step-b are labelled as CS-001-C2 to CS-017-C2 and the extracts obtained with extractive value ranging from 12% to 53%. The results are shown in FIG. 1b, 2b, FIG. 3b, 4b and FIG. 5b.

The obtained fractions in the columns in step-c, as depicted in as depicted in the FIG. 1b, 2b, FIG. 3b, 4b and FIG. 5b are labelled as CS-001-C1(C2)-F1(F2)(F3)(F3)(F4)(F5)(F6)(F7) to CS-017-C1(C2)-F1(F2)(F3)(F3)(F4)(F5)(F6)(F7). The process provided the CBD-5 and THC-5 enriched fractions ranging from 10 to 99%.

The fractions CS-001-C1(C2)-F1(F2)(F3)(F3)(F4)(F5)(F6)(F7) to CS-017-C1(C2)-F1(F2)(F3)(F3)(F4)(F5)(F6)(F7) wherein CBD and THC ranging from 10 to 99% obtained from column chromatography were mixed using appropriate solvents such as ethyl acetate, methanol, ethanol, iso-propanol, etc. and then dried to obtained the blend (CS-001-B1(B2)(B3) to CS-017-B1(B2)(B3) containing both CBD and THC ranging from 5 to 49.5%.

In another embodiment of the present invention provides a process for the preparation of a phytocannabinoids enriched blend containing both CBD and THC in the range of 5% to 49.5% comprising the steps of:
a. combining of CBD and THC fractions in the range of 10 to 99%;
b. mixing the above fraction(s) in step-a with organic solvent, selected from ethyl acetate, methanol, ethanol, iso-propanol, etc;
c. drying the pure fraction obtained from step b to obtain the enriched blend.

In further embodiment of the present invention, a pharmaceutical formulation comprising a therapeutically amount of phytocannabinoids enriched blend consisting of CBD and THC with different strength in the range of 2.5 to 20% prepared and at least 20 to 30 weight equivalent of one pharmaceutically acceptable excipient or a vehicle.

In another embodiment, the present invention relates to methods of treating method for the treatment of a condition or disease selected from the group consisting of management of cancer pain, general pain, comprising administering to a patient in need thereof a therapeutically effective amount of CBD and THC with different strength ranging from 2.5 to 20% and at least one pharmaceutically acceptable excipient or a vehicle.

As the present invention is towards the development of product for the management of pain and accordingly the fraction with varied range of enriched fractions of CBD and THC were combined and studied for formulation and pharmacological activity against appropriate pain models.

The present invention refers to a “pharmaceutically acceptable excipient” or “vehicle” that may be selected from nutriose FB06, dextrin and maltodextrin.

The use of nutriose and malodextrin (which is a soluble fiber, a modified maltodextrin, gluten-free vehicle with high chemical stability and better powder properties) provided acceptable look and free-flowing properties to the phytocannabinoids enriched blend. The formulation having CBD and THC with different strength ranging from 2.5 to 20% were prepared with nutriose FB06, dextrin and maltodextrin.

The phytocannabinoids enriched blend (CS-001-B1(B2)(B3) to CS-017-B1(B2)(B3) were mixed with appropriate acceptable excipients or vehicle such as nutriose FB06, dextrin and maltodextrin and developed a free-flowing powder form which may be packed in any of the oral dosage forms such as hard capsules, soft capsules, swallowable tablets, orally dispersible tablets, chewable tablets, effervescent tablets, lozenges, orally dissolving films, blends, granules.

In another embodiment of the present invention involved the use of phytocannabanoids enriched fraction CS-001-C1(C2)-F1(F2)(F3)(F3)(F4)(F5)(F6)(F7) to CS-017-C1(C2)-F1(F2)(F3)(F3)(F4)(F5)(F6)(F7) and phytocannabanoids enriched blend (CS-001-B1(B2)(B3) to CS-017-B1(B2)(B3) for the treatment of pain in cancer chemotherapy (paclitaxel (PT)) induced neuropathic pain in BALB/C mice using Randall Selitto Test, wherein the phytocannabanoids enriched fraction and blend shown significant increased mechanical threshold and attenuated PT induced neuropathic pain.

In another embodiment of the present invention involved the use of phytocannabanoids enriched fraction CS-001-C1(C2)-F1(F2)(F3)(F3)(F4)(F5)(F6)(F7) to CS-017-C1(C2)-F1(F2)(F3)(F3)(F4)(F5)(F6)(F7) and phytocannabanoids enriched blend (CS-001-B1(B2)(B3) to CS-017-B1(B2)(B3) for the treatment of pain in tail flick assay for the assessment of thermal nociceptive response in BALB/c mice, wherein the phytocannabanoids enriched fraction and blend shown significant increased the latency time (analgesic effect) in response to thermal stimuli.

In another embodiment of the present invention involved the use of phytocannabanoids enriched fraction CS-001-C1(C2)-F1(F2)(F3)(F3)(F4)(F5)(F6)(F7) to CS-017-C1(C2)-F1(F2)(F3)(F3)(F4)(F5)(F6)(F7) and phytocannabanoids enriched blend (CS-001-B1(B2)(B3) to CS-017-B1(B2)(B3) for the treatment of pain in acetic acid induced writhing model in BALB/c mice, wherein the phytocannabanoids enriched fraction and blend shown significant decreases in writhes.

Having described the invention with reference to certain aspects embodiments, embodiments will become apparent to one skilled in the art from consideration of the specification. The invention is further defined by reference to the following examples describing the preparation of enrichment of phytocannabniods and its invivo efficacy studies. It will be apparent to those skilled in the art that many modifications, both to materials and methods, may be practiced without departing from the scope of the invention.

### Example 1: Preparation of Cannabis Extracts with Different Solvents

The authentic plant material of Cannabis sativa (aerial part) was collected from permitted land area at Chatha farm, IIIM Jammu (Jammu and Kashmir State, India). The plant was pharmacognostically/taxonomically characterized, and a voucher specimen (bearing number: 25119, Dated: 15 Jun. 2021) was deposited in the Janaki Ammal Herbarium at the CSIR-IIIM Jammu.

The steps involved:
i. Extraction with hexane (CS-001):
   Grounded plant material (aerial part, 50 g) was mixing with hexane in the proportional ratio of 1:8 (w/v) in a closed extraction vessel and stirring for a period of 8 hours at room temperature. This process was repeated two or three more times with similar conditions and the extract was collected in a container and concentrated by vacuo rota-evaporator at 40-70° C. which after drying gave 4.126 g of material CS-001-C1 (E.V.: 8.2% w/w). The HPLC quantification analysis using method defined in the Example 7 (c) with contents of CBD, CBDA, THC and THCA were 6.6, 3.7. 13.2 and 3.9% w/w, respectively, based on dry weigh basis. The results are shown in the FIG. 1b.
ii. Extraction with ethyl acetate (CS-002):
   Grounded dried plant material (aerial part, 50 g) was extracted with ethyl acetate as per steps mentioned in step (i). Wherein, after drying gave 4.429 g of material CS-002-C1 (E.V.: 8.8% (w/w)). The HPLC quantification analysis using method defined in the Example 7 (c) with contents of CBD, CBDA, THC and THCA were 5.5, 3.7. 10.2 and 3.5% (w/w), respectively, based on dry weigh basis. The results are shown in the FIG. 1b.
iii. Extraction with dichlormethane (CS-003):
   Grounded dried plant material (aerial part, 50 g) was extracted with dichloromethane as per steps mentioned in step (i). Wherein, after drying gave 4.100 g of material CS-003-C1(E.V.: 8.2% (w/w)). The HPLC quantification analysis using method defined in the Example 7(c) with contents of CBD, CBDA, THC and THCA were 6.1. 3.2, 11.0 and 2.9% (w/w), respectively, based on dry weigh basis. The results are shown in the FIG. 1b.
iv. Extraction with chloroform (CS-004):
   Grounded dried plant material (aerial part, 50 g) was extracted with chloroform as per steps mentioned in the step (i). Wherein, after drying gave 5.517 g of material CS-004-C1 (E.V.: 11.0% (w/w)). The HPLC quantification analysis using method defined in the Example 7 (c) with contents of CBD, CBDA, THC and THCA were 5.0, 3.4. 9.8 and 3.2% (w/w), respectively, based on dry weigh basis. The results are shown in the FIG. 1b.
v. Extraction with iso-propyl alcohol (IPA) (CS-005):
   Grounded dried plant material (aerial part, 50 g) was extracted with IPA as per steps mentioned in the step (i). Wherein, after drying gave 5.176 g of material CS-005-C1 (E.V.: 10.3% (w/w)). The HPLC quantification analysis using method defined in the Example 7(c) with contents of CBD, CBDA, THC and THCA were 7.0, 4.1, 10.5 and 3.7% (w/w), respectively, based on dry weigh basis. The results are shown in the FIG. 1b.
vi. Extraction with methanol (CS-006)
   Grounded dried plant material (aerial part, 50 g) was extracted with methanol as per steps mentioned in the step (i). Wherein, after drying gave 8.027 g of material (E.V.: 16.0% (w/w)), CS-006-C1. The HPLC quantification analysis using method defined in the Example 7(c) with contents of CBD, CBDA, THC and THCA were 2.5, 1.6, 5.0 and 1.2% (w/w), respectively, based on dry weigh basis. The results are shown in the FIG. 1b.
vii. Extraction with ethanol (CS-007):
   Grounded dried plant material (aerial part, 50 g) was extracted with Ethanol as per steps mentioned in the step (i). Wherein, after drying gave 7.0 g of material (E.V.: 14.0% (w/w)), CS-007-C1. The HPLC quantification analysis using method defined in the Example 7(c) with contents of CBD, CBDA, THC and THCA were 3.3. 1.1. 9.6 and <LOQ % (w/w), respectively, based on dry weigh basis. The results are shown in the FIG. 1b.
viii. Extraction with mixture of ethyl acetate and ethanol (CS-008):
   Grounded dried plant material (aerial part, 50 g) was extracted with mixture of Ethyl acetate and Ethanol (with portion of 1:9 (v/v)) as per steps mentioned in the step (i). Wherein, after drying gave 7.056 g of material (E.V.: 14.1% (w/w)), CS-008-C1. The HPLC quantification analysis using method defined in the Example 7 (c) with contents of CBD, CBDA, THC and THCA were 4.6, 3.8, 9.6 and 3.2% (w/w), respectively, based on dry weigh basis. The results are shown in the FIG. 1b.
ix. Extraction with mixture of ethyl acetate and methanol (CS-009):
   Grounded dried plant material (aerial part, 50 g) was extracted with mixture of ethyl acetate and methanol (with portion of 1:9 (v/v)) as per steps mentioned in the step (i). Wherein, after drying gave 6.818 g of material (E.V.: 13.6% (w/w)) CS-009-C1. The HPLC quantification analysis using method defined in the Example 7 (c) with contents of CBD, CBDA, THC and THCA were 3.5, 2.8, 7.2 and 2.2% (w/w), respectively, based on dry weigh basis. The results are shown in the FIG. 1b.
x. Extraction with mixture of chloroform and ethanol (CS-010):
   Grounded dried plant material (aerial part, 50 g) was extracted with mixture of chloroform and ethanol (with portion of 1:9 (v/v)) as per steps mentioned in the step (i). Wherein, after drying gave 7.532 g of material (E.V.: 15.0% (w/w)) CS-010-C1. The HPLC quantification analysis using method defined in the Example 7 (c) with contents of CBD, CBDA, THC and THCA were 2.7, 2.7, 5.8 and 1.7% (w/w), respectively, based on dry weigh basis. The results are shown in the FIG. 1b.
xi. Extract with mixture of water and ethanol (CS-011):
   Grounded dried plant material (aerial part, 50 g) was extracted with mixture of water and ethanol (with portion of 1:9) as per steps mentioned in the step (i). Wherein, after drying gave 8.854 g of material (E.V.: 17.7% (w/w)), CS-011-C1. The HPLC quantification analysis using method defined in the Example 7 (c) with contents of CBD, CBDA, THC and THCA were 3.6, 2.3, 7.6 and 1.6% (w/w), respectively, based on dry weigh basis. The results are shown in the FIG. 1b.

### Example 2: Enrichment of Cannabis Extract Using Different Solid Supports

In one of the preferred embodiments of the present invention, further enrichment of cannabis extract was done by using solid support followed by extraction as giving below:
a. Treatment with solid support (Type 1: silica gel 60-120 mesh size) (CS-012)
   Grounded dried plant material (aerial part, 50 g) was extracted with solvent used in the step vii ie ethanol following the procedure of step (i) of Example 1. This extract (CS-012-C1) (7 g) was treated with type 1 solid support (silica gel 60-120 mesh size) with proportions of 1:2.5 (w/w) in a closed container and further extracted with mixture of polar and non-polar solvents like ethyl acetate and hexane with proportions of 1:9 (v/v) after continuous stirring for a period of 4 hours as depicted in the FIG. 2a. After first cycle, the treated extract was repeated with same mixture of solvent for 2 to 4 times and the collected organic layer was evaporated with vacuo rota-evaporator at 70° C. After evaporation, 3.66 g of material obtained CS-012-C2 with extractive value of 52.3% (w/w. w.r.t. CS-012-01) with content of CBD, CBDA, THC and THCA were 13.1, 1.3, 13.1 and <LOQ, respectively. These phytocannabinoids were estimated/analyzed by using HPLC. The results are depicted in the FIG. 2b.
b. Treatment with solid support (Type 2: silica gel 100-200 mesh size) (CS-013)
   Grounded dried plant material (aerial part, 50 g) was extracted with solvent used in the step vii i.e. ethanol and following the procedure of step (i) of Example 1. This extract (CS-013-C1) (7.1 g) was treated with type 2 solid support (silica gel 100-200 mesh size) as procedure mentioned in the step a of Example 2 and as depicted in FIG. 2a. After evaporation (at 70° C.), 2.30 g of material obtained CS-013-C2 with extractive value of 32.3% (w/w. w.r.t. CS-013-01) with content of CBD, CBDA, THC and THCA were 10.7. 2.4, 16.6 and 0.6% (w/w) respectively. The results are depicted in the FIG. 2b.
c. Treatment with solid support (Type 3: silica gel 230-400 mesh size) (CS-014)
   Grounded dried plant material (aerial part, 50 g) was extracted with solvent used in the step vii i.e. ethanol and following the procedure of step (i) of Example 1. This extract (CS-014-C1) (6.9 g) was treated with type 3 solid support (silica gel 230-400 mesh size) as procedure mentioned in the step a of Example 2 and as depicted in FIG. 2a. After evaporation (at 70° C.), 1.94 g of material obtained CS-014-C2 with extractive value of 28.1% (w/w. w.r.t. (CS-014-01) with content of CBD, CBDA, THC and THCA were 8.9, 2.4, 15.5 and 0.4% (w/w), respectively. The results are depicted in the FIG. 2b.

### Example 3: Enrichment of Phytocannabniods, Using Different Mixture of Polar and Non-Polar Solvents

i. Mixture of ethyl acetate and hexane: (CS-015-C2a)
Grounded dried plant material (aerial part, 150 g) was extracted with solvent used in the step vii i.e. ethanol and following the procedure of step (i) of Example 1. In this experiment the obtained extractive value was found to be E.V. 16.0% (w/w) and 24.1 g of extract (CS-015-C1) was received. Out of 24 g. 8.2 g was taken for further enrichment process as mentioned in the procedure in the step a of Example 2 by treated with type 1 solid support (silica gel 60-120 mesh size) and extracted with mixture of polar and non-polar solvent like ethyl acetate and hexane. After evaporation (at 40° C.), 2.40 g of material CS-015-C2a was obtained. The extractive value (CS-015-C2a) was found to be 29.2% (w/w) with content of CBD, CBDA, THC and THCA were 7.5, 7.0, 19.2 and 5.8% (w/w), respectively. The results are depicted in the FIG. 3b.
   ii. Mixture of dichloromethane: hexane: (CS-015-C2b)
7.9 g of extract was taken from the CS-015-C1 was treated with type 1 solid support (silica gel 60-120 mesh size) and extracted with mixture of dichloromethane and hexane as procedure mentioned in step a of Example 2. After evaporation (at 40° C.), 1.046 g of material (CS-015-C2b) was obtained The extractive value (CS-015-C2b) was found to be 13.2% (w/w) with content of CBD, CBDA, THC and THCA were 10.8, 4.5, 24.3 and 2.1% (w/w), respectively. The results are depicted in the FIG. 3b.
   iii. Mixture of chloroform: hexane mixture (CS-015-C2c)
8.0 g of extract was taken from the CS-015-C1 and treated with type 1 solid support (silica gel 60-120 mesh size) and extracted with mixture of chloroform and hexane as procedure mentioned in step a, of Example 2. After evaporation (at 40° C.), 1.082 g of material CS-015-C2c was obtained. The extractive value (CS-015-C2c) was found to be 13.5% (w/w) with content of CBD, CBDA, THC and THCA were 11.6, 4.7, 26.0 and 2.2% (w/w), respectively. The results are depicted in the FIG. 3b.

### Example 4: Enrichment of Phytocannabinoids and Their Isolation

1. Procedure for Enrichment of Phytocannabinoids and their isolation (CS-16) was illustrated in following steps:
   a. As depicted in the FIG. 4a, the grounded plant material (aerial part, 25 Kg) extracted with ethanol (1:8 (w/v)) as per procedure mentioned in the Example 1, step (i). The extract was dried/evaporated at 70° C. through tray dryer. The extractive value of alcoholic extract CS-016-C1 was found to be 16% (w/w) on dried weight basis. The percentage content of CBD and THC was approximately 2.9% (w/w) and 2.2% (w/w), respectively, and same was quantified by HPLC method. The results are depicted in the FIG. 4b.
   b. Above concentrated alcoholic extract CS-016-C1 (4 Kg) treated with solid support Type 1 (silica gel 60-120 mesh size) in a proportion of 1:2.5% (w/w) and further extracted by using mixture of polar and non-polar solvents (1:9 (v/v)) as mentioned procedures at Example 2 at step (a) or Example 3 at step (i) for enrichment of phytocannabinoids. The eluent solvent evaporated at 70°° C. through tray dryer. After evaporation, 1.5 Kg of material CS-016-C2 was obtained. The extractive value of enriched extract (CS-016-C2) was found to be 37.5% (w/w), on dried weight basis. The percentage content of CBD and THC was approximately 12.4% (w/w) and 9.1% (w/w) respectively and same was quantified by HPLC method. The results are depicted in the FIG. 4b. The selection of solvent system is based upon the extractive value, phytocannabinoids percentage and most safe and acceptable solvent systems. The hexane:ethylacetate solvent system have given very high extractive value, therefore, the hexane:ethylacetate solvent system has been selected for further step.
   c. To further enrichment of above-mentioned enriched extract CS-016-C2 (1.5 Kg) run over the column chromatography contains solid support type 2 (100-200 mesh size). The phytocannabinoids were eluted by ethyl acetate and hexane with proportions of 1-5% (v/v). In this process, five fractions were collected, wherein first fraction CS-016-C2-F1, was obtained as 100 g material. In second fraction CS-016-C2-F2 contains almost 1:1 proportion of CBD and THC content i.e., 18% (w/w) CBD and 17.3% (w/w) THC and received an amount of 245 g of enriched extract. In third fraction CS-016-C2-F3, in which 19% (w/w) THC and 1% (w/w) CBD presented and received an amount of 175 g of material. In fourth fraction CS-016-C2-F4, contain 0.12% (w/w) CBD and 0.05% (w/w) THC and received an amount of 65 g. In last fraction, 1 kg of undesired phytoconstituents like chlorophyll, fatty acids and alkaloids etc., material was received. All these five fractions were collected in a different container's and concentrated at 40-70° C. through vacuo rota-evaporator. The content of phytocannabinoids were estimated by using HPLC method on dried weight basis. The results are depicted in the FIG. 4b.
   d. The above collected second fraction CS-016-C2-F2 (245 g) was further enriched as per procedure mentioned in above step with polarity of 1-2% (v/v) of ethyl acetate and hexane. In this enrichment process, four fractions were again collected. In first fraction CS-016-C2-F2-F1, received an amount of 100 g, wherein phytocannabinoids were not presented and same was analyzed on the basis of TLC. In second fraction CS-016-C2-F2-F2, 30 g of material was received, in which 58% (w/w) of CBD and 0.4% (w/w) of THC content were present. In third fraction CS-016-C2-F2-F3, wherein THC rich fraction 54% (w/w) was collected with an amount of 29 g of material. In last fraction 95 g of unwanted material was received i.e., phytoconstituents like chlorophyll, fatty acids and alkaloids etc. All these four fractions were collected in a different container's and concentrated at 40-70° C. through vacuo rota-evaporator. The content of phytocannabinoids were estimated by using HPLC method on dried weight basis. The results are depicted in the FIG. 4b.
   e. CBD (58% (w/w) enriched fraction CS-016-C2-F2-F2 (30 g) was further enriched with same solvent system through combi flash chromatography by using normal phase. In this enrichment process, three fractions were collected. In first fraction CS-016-C2-F2-F2-F1, collected the non phytocannabinoids. In second fraction CS-016-C2-F2-F2-F2 (15 g) of material was obtained with desired phytocannabinoids i.e. >83% (w/w) of CBD content was collected. In last fraction, unwanted material was received. The results are depicted in the FIG. 4b.
   f. THC (54% (w/w)) enriched fraction CS-016-C2-F2-F3 was further enriched, using same solvent system through combi flash chromatography by using normal phase. In this enrichment process, three fractions were collected. In first fraction CS-016-C2-F2-F3-F1, collected the non phytocannabinoids. In second fraction CS-016-C2-F2-F3-F2 (16 gm) of material was obtained with desired phytocannabinoids i.e. >83% (w/w) of THC content was collected. In last fraction, unwanted material was received. The results are depicted in the FIG. 4b.
   g. Further enrichment of the desired phytocannabinoids was done through semi preparative HPLC using reverse phase column (RP-C18) with mixture of acetonitrile and water by isocratic technique. In isolation process of CBD fraction of CS-016-C2-F2-F2-F2 with quantity of 4 mg was taken, wherein 2 mg of CBD with purity of 99.0% (w/w) collected at retention time 25 mins and for isolation of THC using fraction of CS-016-C2-F2-F3-F2 with quantity of 4 mg was taken, wherein 2 mg of THC with purity 98.2% w/w was collected at 40 mins retention time. These collections were concentrated through vacuo rota-evaporator. The results are depicted in the FIG. 4b.
2. Procedure for Enrichment of Phytocannabinoids and their isolation (CS-17) was illustrated in following steps:
   a. As depicted in the FIG. 4a, the grounded plant material (aerial part, 25 Kg) extracted with ethanol (1:8 (w/v)) as per procedure mentioned in the Example 1, step (i). The extract was dried/evaporated at 70° C. through tray dryer. The extractive value of alcoholic extract CS-017-C1 was found to be 16% (w/w) on dried weight basis. The percentage content of CBD and THC was approximately 3.4% (w/w) and 15% (w/w) respectively, and same was quantified by HPLC method. The results are depicted in the FIG. 5b.
   b. Above concentrated alcoholic extract CS-017-C1 (4 Kg) treated with solid support Type 1 (silica gel 60-120 mesh size) in a proportion of 1:2.5% (w/w) and further extracted by using mixture of polar and non-polar solvents (1:9 (v/v)) as mentioned procedures at Example 2 at step (a) or Example 3 at step (i) for enrichment of phytocannabinoids. The eluent solvent evaporated at 70° C. through tray dryer. After evaporation, 1.5 Kg of material CS-017-C2 was obtained. The extractive value of enriched extract (CS-017-C2) was found to be 37% (w/w), on dried weight basis. The percentage content of CBD and THC was approximately 6.5% (w/w) and 26% (w/w) respectively and same was quantified by HPLC method. The results are depicted in the FIG. 5b. To further enrichment of above-mentioned enriched extract CS-017-C2 (1.5 Kg) run over the column chromatography contains solid support type 2 (100-200 mesh size). The phytocannabinoids were eluted by ethyl acetate and hexane with proportions of 1-5% (v/v). In this process, seven fractions were collected, wherein first fraction CS-017-C2-F1, was obtained as 100 g material. In second fraction CS-017-C2-F2 contains CBD and THC content i.e., 31% (w/w) CBD and 3% (w/w) THC and received an amount of 200 g of enriched extract. In third fraction CS-017-C2-F3, in which 28% (w/w) CBD and 31% (w/w) THC presented and received an amount of 120 g of material. In fourth fraction CS-017-C2-F4, contain 4% (w/w) CBD and 13% (w/w) THC and received an amount of 50 g. In fifth fraction CS-017-C2-F5, contain 3% (w/w) CBD and 5% (w/w) THC and received an amount of 15 g. In sixth fraction CS-017-C2-F6, contain 1% (w/w) CBD and 2% (w/w) THC and received an amount of 180 g. In last fraction, 832 g of undesired phytoconstituents like chlorophyll, fatty acids and alkaloids etc., material was received. All these five fractions were collected in a different container and concentrated at 40-70° C. through vacuo rota-evaporator. The content of phytocannabinoids were estimated by using HPLC method on dried weight basis. The results are depicted in the FIG. 5b.

### Example 5: Mixing of Phytocannabinoids Enriched Fractions

a. One of the preferred embodiments of the present invention, is the preparation of CBD-THC composition for formulation. The CBD-enriched fraction, 1 g of CS-016-C2-F2-F2 and THC-enriched fraction, 1 g of CS-016-C2-F2-F3 mixed together with same quantity by using ethanol as solvent and then dried under vaccum. The obtained gummy material CS-016-B1 was then analyzed for CBD and THC content through HPLC validated method, wherein CBD:THC content having 27% (w/w) and 25% (w/w) respectively. These results are depicted in the FIG. 6.
b. In one of the preferred embodiments of the present invention, preparation of CBD-THC composition for formulation. The CBD-enriched fraction, 1 g of CS-016-C2-F2-F2-F2 and THC-enriched fraction, 1 g of CS-016-C2-F2-F3-F2 mixed together with same quantity by using ethanol as solvent and then dried under vaccum. The obtained gummy material (CS-016-B2) was then analyzed for CBD and THC content through HPLC validated method, wherein CBD: THC content having 40.2% (w/w) and 36% (w/w) respectively. These results are depicted in the FIG. 6.
c. In one of the preferred embodiments of the present invention, preparation of CBD-THC composition for formulation. The CBD-enriched fraction, 2 g of CS-016-C2-F2-F2-F2 and THC-enriched fraction, 1 g of CS-016-C2-F2-F3-F2 mixed together with same quantity by using ethanol as solvent and then dried under vaccum. The obtained gummy material (CS-016-B3) was then analyzed for CBD and THC content through HPLC validated method, wherein CBD: THC content having 54% (w/w) and 26.7% (w/w) respectively. These results are depicted in the FIG. 6.
d. In one of the preferred embodiments of the present invention, preparation of CBD-THC composition for formulation. The CBD: THC enriched fraction, 120 g of CS-017-C2-F3 and mixing with enriched fraction, 10 g of CS-016-C2-F2-F2-F2 by using ethanol as solvent and then dried under vaccum. The obtained gummy material (CS-017-B1) was then analyzed for CBD and THC content through HPLC validated method, wherein CBD:THC content having 34% (w/w) and 29% (w/w) respectively. These results are depicted in the FIG. 6.

### Example 6: In-Vivo Efficacy Studies

a. In-Vivo Efficacy Study in Cancer Pain Model Using the Randall Selitto Test for Assessment of Mechanical Hyperalgesia in Male BALB/c Mice

### Methods

Animals: Male BALB/C mice of weight range 25-30 g were used for evaluation of CS-016-B1 in paclitaxel (PT) induced neuropathic pain. Animals were housed under a 12-hour light/dark cycle in a temperature (65-75° F.; ^{˜}18-23° C.) and humidity-controlled (40-60%) environment. They were supplied with free access to the food and water (ad libitum). Prior to initiation of study all the animals were acclimatized for one week under standard laboratory conditions, animals were drug naive with no prior procedures performed. All testing were performed from 1 to 4 p.m. Mice were randomized in groups based on their body weights for all behavioural assays and tests were performed by an experimenter blinded to the treatment groups. All experiment protocols were approved by the Institutional Animal Ethics Committee (IAEC) (IAEC approval no.-222/78/2/2021), and followed the Committee for the Purpose of Control and Supervision of Experiments on Animals (CPCSEA; Ministry of Environment and Forest, Government of India) guidelines for animal care.

Drugs and chemicals: CS-016-B1 was prepared in-house by Medicinal chemistry Division (CSIR-IIIM). Paclitaxel was provided as gift sample by Konark Invacare Pvt. Ltd., Mumbai. Pregabalin was purchased from Leeford Healthcare Ltd. Crempohor EL was procured from Sigma Aldrich (Mumbai, India).

Development of neuropathic pain in mice: Neuropathic pain in BALB/C mice was induced by intraperitoneal (i.p.) administration of Paclitaxel (PT) at 2 mg/kg for consecutive 5 days (Sci. Rep., 2014, 4, 1). Before paclitaxel injection baseline values of pain threshold for all the animals were recorded. For neuropathic pain induction, the pain behavioural assessment was carried out post paclitaxel injection (Day-3, Day-5). After confirming the pain induction with decreased nociceptive threshold to baseline values dosing of CS-016-B1 (15, 30, 60 mg/kg. i.p.) (Neuropsychopharmacology., 2019, 45(7), 1105), (Pain, 2017, 158(12), 2452), (Br. J. Pharmacol., (2014), 171(3), 636), (J. Exp. Med., 2012, 209(6), 1121) and PreG (10 mg/kg. i.p.) initiated (Sci. Rep., 2018, 8(1), 16402, 1), (Curr. Neuropharmacol., 2014, 12(1), 44) then pain assessment was carried out at different time points like 5^{th}, 10^{th}, 15^{th} days post dosing.

Experimental protocol: The present study was comprised of five experimental groups. Each group includes five BALB/C mice (n=5). Paclitaxel (PT) (2 mg/kg) was administrated intraperitoneally to all the groups for 5 days. Group I (Control): Mice were subjected to vehicle (0.5% Cremophor EL and 0.5% absolute ethanol, volume make up with normal saline) administration. CS-016-B1 and PT were dissolved in the same vehicle. Group II (CS-016-B1, 15 mg/kg, i.p.): Mice received an intraperitoneal dose of CS-016-B1 (15 mg/kg) for 15 days (from day 6). Group III (CS-016-B1, 30 mg/kg, i.p.): Mice received an intraperitoneal dose of CS-016-B1 (30 mg/kg) for 15 days (from day 6). Group IV (CS-016-B1, 60 mg/kg): Mice received an intraperitoneal dose of CS-016-B1 (60 mg/kg) for 15 days (from day 6). Group V (Pregablin; 10 mg/kg, i.p.): Mice received an intraperitoneal dose of PreG (10 mg/kg) for 15 days from day 6. All five groups were involved in the assessment of pain behavior assessment. Pain threshold was assessed using Randall Selitto for assessment of mechanical hyperalgesia at different time points like 0, 5th, 10th, and 15th days post dosing. The end of the study period i.e., 16th day mice were sacrificed after behavioral assessments.

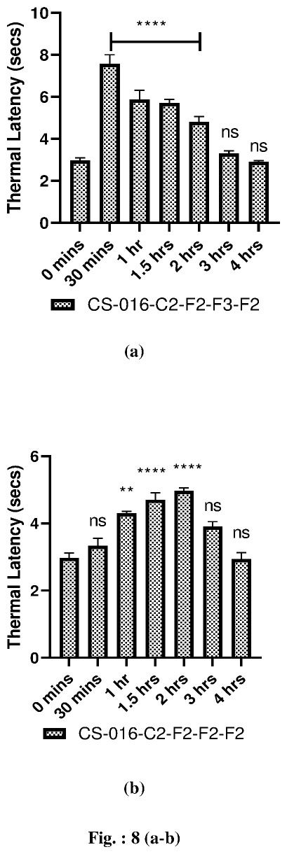

Randall Selitto Test: The nociceptive withdrawal threshold was assessed by using the Randall-Selitto algesimeter (Analgesy-Meter, Ugo Basile). This test involved application of an increasing mechanical force to the surface of the paw or tail until withdrawal or vocalization occurs (J. Neurotrauma., 2012, 29, 898). Because no response from the animals was observed on the medial portion of dorsal and plantar paw surfaces so the pressure was applied to the tail. During the test, tail of each mouse was placed and immobilized to the tip of the algesimeter followed by application of an increasing mechanical force until it resulted into vocalization or a withdrawal response. The point of application was marked with ink marker in order to provide pressure to the same location over repeated trials, three trials were carried out for each animal. The maximum force applied to each mice was limited to 125 g to avoid skin damage (Front. Mol. Neurosci., 2017, 8, 16402(1)). Mechanical threshold (g) was calculated for each mouse by recording scale values of apparatus (Scale value 2=5 g) (FIG. 7).

Analgesic treatments and drug used: Pregabalin was dissolved in 5% Tween 80 in saline formulated for intraperitoneal (i.p.) delivery of 10 mg/kg. CS-016-B1 was prepared in 0.5% Cremophor EL and 0.5% absolute ethanol to 15, 30, 60 mg/kg concentrations, volume make up with normal saline. Dose volume administered was 10 ml/kg/mice.

Statistical analysis: The behavioural data are indicated as mean±standard deviation (SD). Data of pain behavioural assessments were analyzed using Graph pad prism software (8.0.2). Two-way analysis of variance (ANOVA) was applied with post-hoc Tukey test at 95% CI of difference. A probability value i.e., (p<0.05) was considered statistically significant. ****p<0.0001; ***p<0.005; **p<0.05; *p<0.05

### Results

Analysis of effect of paclitaxel, CS-016-B1 and pregabalin on neuropathic pain in the Randall-Selitto test: The intraperitoneal administration of paclitaxel (2 mg/kg) resulted in a significant reduction in mechanical threshold in all the groups that received the drug when compared with the baseline data (FIGS. 7a and 7b). Furthermore, administration of CS-016-B1 at all doses (15, 30 and 60 mg/kg.i.p.) significantly increased mechanical threshold and attenuated PT induced neuropathic pain. The readings were taken at three-time intervals including Day 5. Day 10 and Day 15. At Day 05, the animals treated with different doses of CS-016-B1 displayed highly improved response to the mechanical pressure. The antihyperalgesic effect of CS-016-B1 at 15 mg/kg and higher doses was almost equal to pregabalin at 10 mg/kg (FIG. 7c). The antihyperalgesic effect of CS-016-B1 was sustained even at day 10 and day 15 at all the treatment doses (FIGS. 7d and 7e).

b. In-vivo efficacy study in pain model using the tail flick Assay for assessment of thermal nociceptive response in female BALB/c mice

Tail Flick Test: Prior to starting the test, the intensity of the beam was adjusted to produce a latency of approximately 3-6 sec. The was performed three times for each animal, at 3- to 5-min intervals, and the average of the three times recorded as the average reaction time (seconds). The light beam was delivered about 15 mm from the tip of the tail for mice. To minimize the chance of tissue trauma from heat exposure, a cut-off time of 10 sec was established, at which time the animal was removed from the test (Curr. Protoc. Neurosci., 2007, 41, 8.9.1). Mice were treated with diclofenac sodium (50 mg/kg. i.p.), a standard analgesic drug, CS-016-C2-F2-F2-F2 (10 mg/kg. p.o), CS-016-C2-F2-F3-F2 (10 mg/kg, p.o). and CS-016-B2 (10 mg/kg, p.o) each group contained three animals (n=3). Average reaction time against radiant stimuli was noted for each group at 0-, 30- and 60-mins post drug administrations.

### Results and Discussion of Tail Flick

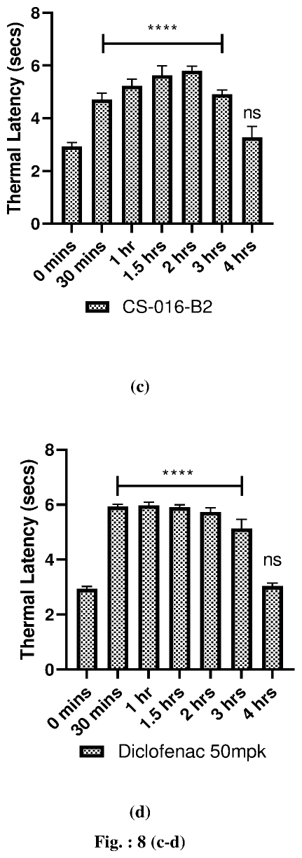

Tail Flick Test, Oral treatment of animals with CS-016-C2-F2-F3-F2 (10 mg/kg) showed significantly increased reaction latency time from 2.97 seconds baseline at 0 h to 7.57 seconds CS-016-C2-F2-F3-F2, 10 mg/kg) to thermal stimuli after 30 mins of treatment (FIG. 8a). The effect of CS-016-C2-F2-F3-F2 on reaction latency time sustained till 3 h of treatment with gradual reduction, however, it became almost equal to baseline at 4 h (FIG. 8a). Effect of CS-016-C2-F2-F2-F2 alone (10 mg/kg) on latency time was clearly observed after one hour of treatment (4.3 seconds), which sustained till 3 h, and at 4 h the reaction time of animals was similar to that of baseline (FIG. 8b). The group treated with CS-016-B2 (10 mg/kg) showed significantly enhanced latency time of 4.7 seconds, which was further increased to 5.8 seconds after 2 h treatment. This effect was declined to 3.27 seconds even after 4 h of treatment (FIG. 8c). The animals treated with the standard drug diclofenac sodium (50 mg/kg) showed enhanced latency time through 3 h after treatment, however, after 4 h of treatment these animals showed the latency time comparable to that of baseline data (FIG. 8d). The treatment with combination of CS-016-B2 (10 mg/kg) appeared to have almost similar analgesic effect as that of CS-016-C2-F2-F3-F2 (10 mg/kg) except after the 30 minutes treatment, which is an advantage over psychotropic CS-016-C2-F2-F3-F2 at higher doses.

Tail Flick Assay in Female BALB/c Mice. The data presented here show the effect of CS-016-C2-F2-F2-F2, CS-016-C2-F2-F3-F2, CS-016-B2 and the standard drug diclofenac sodium on the pain behaviour of mice. a: Thermal latency of mice post CS-016-C2-F2-F3-F2 (10 mpk), b: Thermal latency of mice post CBD (10 mg/kg) administration administration, c: Thermal latency of mice post CS-016-B2 (10 mg/kg) administration, d: Thermal latency of mice post diclofenac (50 mg/kg) administration. The data are mean of three animals (n=3)±standard error mean (SEM). The data were analysed using graph-pad prism software (8.0.2). Two-way analysis of variance (ANOVA) was applied with post-hoc Tukey test at 95. % CI of difference.

c. In-Vivo Efficacy for the Assessment of Pain Using Acetic Acid Induced Writhing in Femal BALB/c Mice

Acetic Acid induced Writhing: Acetic acid-induced writhing test was performed in BALB/C mice. Briefly. mice were treated with diclofenac sodium (50 mg/kg, i.p.), a standard analgesic drug, CS-016-C2-F2-F2-F2 (10 mg/kg. p.o). CS-016-C2-F2-F3-F2 (10 mg/kg, p.o), CS-016-B2 combination (10 mg/kg, p.o), and one vehicle control group, all the groups contained 3 animals each (n=3). Each group received intra-peritoneal injection of 0.6% acetic acid 30 min after the treatment with drugs including vehicle and the standard. After that number of abdominal stretches i.e. writhes were counted for each group of mice starting from 5 minutes after the injection of acetic acid up to 20 minutes (PLoS One, 2015, 10(9), e0135558).

### Results and Discussion of Acetic Acid Writhing

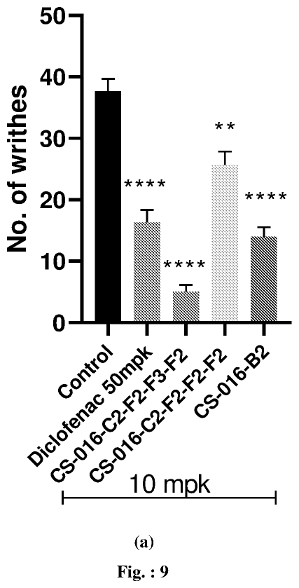

Acetic acid induced writhing: The treatment of mice with 10 mg/kg of CS-016-C2-F2-F3-F2 led to strong suppression of writhes count from about 37 to 05, whereas the effect of CS-016-C2-F2-F2-F2 (10 mg/kg) on acetic acid induced writing was significantly weak as compare to CS-016-C2-F2-F3-F2 or the standard drug diclofenac sodium (FIG. 9a). However, the mice treated with the CS-016-B2 displayed markedly reduced number of writhes when compared to CS-016-C2-F2-F2-F2 alone, moreover, the effect of combination treatment was comparable to that of diclofenac sodium (FIG. 9a). The combination treatment with reduced dose of both CS-016-C2-F2-F3-F2 and CS-016-C2-F2-F2-F2. thus provides a better strategy. where the dose of CS-016-C2-F2-F3-F2 is significantly reduced without compromising with the effect of drug on pain.

Acetic Acid Induced Writhing in Female BALB/c Mice The writhing was induced by intra-peritoneal injection of 0.6% acetic acid 30 min after the treatment with the drugs as shown in the FIG. 9a. The number of abdominal stretches or writhes were counted for each animal in the group starting from 5 minutes after the injection of acetic acid up to 20 minutes. The data are indicated here as mean±standard error mean (SEM), n−=3. Data of pain behavioural assessments were analyzed by using graphpad prism software (8.0.2). One-way analysis of variance (ANOVA) was applied with post-hoc Tukey test at 95. % CI of difference. A probability value i.e., (p<0.0001) was considered statistically significant.

### Example 7: Process of Formulation Development of Phytocannabinoids Enriched Fraction

The phytocannabinoids enriched fraction was further taken up for formulation development, wherein phytocannabinoids enriched fraction formulated with other acceptable excipients such as dextrin/maltodextrin and Nutriose FB06 in oral solid dosage forms (OSD) for administered purpose. The formulation was prepared by taking the fixed amount of phytocannabinoids enriched fraction CS-016-B1 in a glass beaker and then added the fixed amount of Nutriose and then transferred the material into ceramic pestle-mortar and mixed for 30 min to 1 hour till the formation of free-flowing material.
a. In the one of the experiment, phytocannabinoids enriched fraction CS-017-B1 (1 g) was taken in a glass beaker and then added the Nutriose (25.6 g), then transferred into ceramic mortar-pestle and mixed for 30 min to 1 hour till the formation of free-flowing material. The said formulation was then taken up for dissolution study and the content of phytocannabinoids such as THC and CBD were measured using the HPLC method, wherein the percentage of CBD and THC was found to be 1.02 and 0.89%, respectively, with respect to total formulation and the results are depicted in FIG. 6.
b. In the one of the experiments, phytocannabinoids enriched fraction CS-017-B1 (1.2 g) was taken in a glass beaker and then added the maltodextrin (25.6 g), then transferred into ceramic mortar-pestle and mixed for 30 min to 1 hour till the formation of free-flowing material. The said formulation was then taken up for dissolution study and the content of phytocannabinoids such as THC and CBD were measured using the HPLC method, wherein the percentage of CBD and THC was found to be 1.20 and 1.00%, respectively, with respect to total formulation and the results are depicted in FIG. 6.
c. HPLC Method for the analysis and quantification of extract, enriched fraction and formulation: Estimation of markers such as CBD, THC, CBDA and THCA in cannabis based extract, enriched fraction and formulations were performed on RP-18 end capped; 5 μM, 4.6×250 mm. The mobile phase containing buffer (0.1% formic acid in water and 0.1% formic acid in acetonitrile) were used at the flow rate of 1 mL/min at column temperature of 25° C. at 210 nm, 190 nm, 254 nm wavelength range. A volume of 10 μL was injected and a total run time of the assay was 40 mins. The markers were estimated by HPLC using gradient technique. The calibration curve for all the markers compound were established having the concentration range from 1 μg/10 μl to 0.0625 μg/10 μl and the same was used for the estimation and quantification purpose. To estimate the markers in cannabis based extract, enriched fraction and formulation, known quantity of extract, enriched fraction and formulation added to know volume of ethanol and the mixture was sonicated. Supernatant was injected in the HPLC system and analyzed.

1. A process for preparation of pure phytocannabinoids CBD and Δ⁹-THC from Cannabis sativa comprising the steps of:
a. repetitive extraction of dried aerial parts of Cannabis sativa by using polar solvent in the ratio of 1:8 (plant material:solvent (w/v));
b. treating or adsorbing the obtained extract from step-a with silica gel solid support of 60-120 mesh size:
c. extracting the adsorbed silica of step b with a mixture of polar and non-polar solvents at a proportions of 1:9 (v/v) and collecting organic layer:
d. conducting column chromatography of the organic layer obtained in step c using silica gel solid support of 100-200 mesh size, and eluting with ethyl acetate and hexane at a proportion of 1-5% (v/v) to obtain different fractions:
e. collecting the fraction obtained from step d which contains equal proportion of CBD and Δ⁹-THC content and further enriching it by column chromatography using silica gel solid support of 100-200 mesh size, and eluting with ethyl acetate and hexane at a proportion of 1-2% (v/v) of to obtain different fractions:
f. collecting the fractions separately obtained from steps d and e and further enriching the CBD enriched fraction and Δ⁹-THC enriched fraction separately with ethyl acetate and hexane solvent system through combi flash chromatography to obtain different fractions; and
g. collecting the desired phytocannabinoid enriched fractions separately and further enriching through semi preparative HPLC using reverse phase column (RP-C18) with mixture of acetonitrile and water by isocratic technique to obtain pure CBD and Δ⁹-THC.

2. The process as claimed in claim 1, wherein the polar solvent of step a is selected from a group consisting of hexane, ethyl acetate, dichloromethane, chloroform, isopropyl alcohol, methanol, ethanol, ethyl acetate/ethanol, ethyl acetate/methanol, chloroform/ethanol and water/ethanol.

3. The process as claimed in claim 2, wherein the polar solvent of step a is ethanol.

4. The process as claimed in claim 1, wherein the extraction of adsorbed silica with polar and non-polar solvent mixture is repeated 2 to 4 times.

5. The process as claimed in claim 1, wherein the polar and non-polar solvent mixture of step c is selected from ethyl acetate: hexane, dichloromethane: hexane and chloroform: hexane.

6. The process as claimed in claim 5, wherein the polar and non-polar solvent mixture is ethyl acetate: hexane.

7. The process as claimed in claim 1, wherein the purity of CBD is 99.0% (w/w) and purity of Δ⁹-THC is 98.2% w/w.

8. A process for preparation of a phytocannabinoids enriched blend containing CBD and Δ⁹-THC in the range of 5% to 49.5% comprising the steps of:
a. combining of CBD and THC pure fractions as obtained in claim 1:
b. mixing the combined pure fraction of step-a with organic solvent, selected from ethyl acetate, methanol, ethanol and iso-propanol and
c. drying the pure fraction obtained from step b to obtain the enriched blend.

9. A pharmaceutical composition comprising 2.5 to 20% of enriched blend of CBD and Δ⁹-THC as obtained in claim 8 and at least 20 to 30 weight equivalent of one pharmaceutically acceptable excipient or a vehicle for management of pain.

10. The composition as claimed in claim 9. wherein the pharmaceutically acceptable excipient or vehicle is selected from the group consisting of nutriose. dextrin and maltodextrin.
